(19) United States
(12) Patent Application Publication
(10) Pub. No.: US 2007/0032661 A1
(43) Pub. Date: Feb. 8, 2007

(54) Process for the preparation of intermediates of perindopril

(75) Inventors: Narendra Shriram Joshi, Koperkhairane (IN); Buddhavarapu Pattabhi Ramam, Koparkhairane (IN); Arjun Rajaram Bodkhe, Ulashnagar (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/495,102

(22) Filed: Jul. 28, 2006

Related U.S. Application Data
(60) Provisional application No. 60/713,000, filed on Aug. 31, 2005.

(30) Foreign Application Priority Data
Aug. 3, 2005 (IN) 903/MUM/2005

(51) Int. Cl. C07D 209/42

(52) U.S. Cl. 548/492

(57) ABSTRACT

A process for the preparation of (2S, 3aS, 7aS)perhydroindole-2-carboxylic acid is provided comprising (a) esterifying a cis-perhydroindole-2-carboxylic acid with a first alcohol of the formula ROH and a suitable free acid to provide the acid salt (AS) of Formula V: 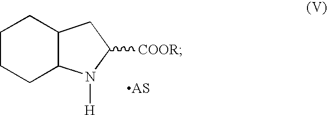
(b) reacting the acid salt of Formula V with a first base to provide a compound of Formula VI: 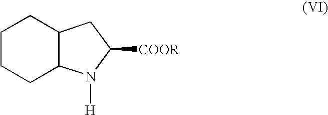
(c) treating the product of step (b) with an L-tartaric containing acid in a second alcohol of the formula ROH to precipitate a compound of Formula VII: 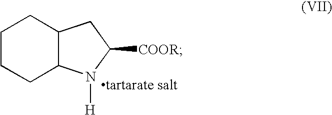
(d) reacting the compound of Formula VII with a second base to provide a compound of Formula II 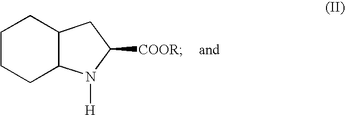 (e) hydrolyzing the compound of Formula II to provide the (2S, 3aS, 7aS)perhydroindole-2-carboxylic acid.

### PRIORITY

This application claims the benefit under 35 U.S.C. §119 to U.S. Provisional Application No. 60/713,000, filed on Aug. 31, 2005, and entitled “PROCESS FOR THE PREPARATION OF INTERMEDIATES OF PERINDOPRIL” and to Indian Provisional Application 903/MUM/2005, filed on Aug. 3, 2005, and entitled “PROCESS FOR THE PREPARATION OF INTERMEDIATES AND USE THEREOF FOR THE PREPARATION OF PERINDOPRIL”, the contents of each of which are incorporated by reference herein.

### BACKGROUND OF THE INVENTION

1. Technical Field

The present invention generally relates to an improved process for the preparation of intermediates for perindopril. More specifically, the present invention relates to an improved process for the preparation of the intermediate (2S,3aS,7aS)-perhydroindole-2-carboxylic acid and use thereof for the preparation of perindopril and derivatives thereof.

2. Description of the Related Art

The present invention is directed to an improved process for the preparation of intermediates for perindopril and perindopril erbumine (also known as (2S,3aS,7aS)-1-[(S)-N-[(S)-1 -carboxy-butyl]alanyl]hexahydro-2-indolinecarboxylic acid, 1-ethyl ester, compound with tert-butylamine (1:1)) of Formula I.

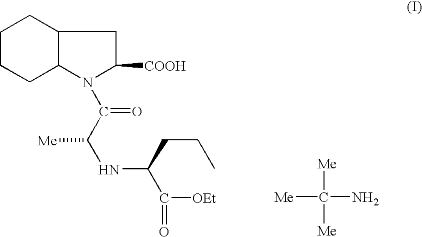

The tert-butylamine salt of perindopril, also known as perindopril erbumine, is the form commercially sold under the trade name Aceon®. Perindopril is the free acid form of perindopril erbumine and is an ethyl ester of a non-sulfhydryl angiotensin-converting enzyme (ACE) inhibitor. Perindopril is a pro-drug and is metabolized in vivo by hydrolysis of the ester group to form perindoprilat, the biologically active metabolite. Perindopril is ordinarily used to treat hypertension.

It is believed that perindoprilat lowers blood pressure primarily through inhibition of ACE activity. ACE is a peptidyl dipeptidase that catalyzes conversion of the inactive decapeptide, angiotensin I, to the vasoconstrictor, angiotensin II. Angiotensin II is a potent peripheral vasoconstrictor, which stimulates aldosterone secretion by the adrenal cortex, and provides negative feedback on renin secretion. Inhibition of ACE results in decreased plasma angiotensin II, leading to decreased vasoconstriction, increased plasma renin activity and decreased aldosterone secretion. The latter results in diuresis and natriuresis and may be associated with a small increase of serum potassium.

European Patent Application No. 0 037 231 discloses 2-carboxyperhydroindole and its ester of Formula II. 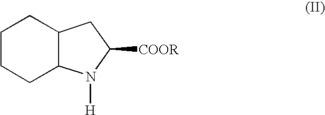
wherein R is hydrogen or a lower alkyl or benzyl group, which is one of the starting reactants for preparing a compound of Formula I.

The compounds of Formula II can exist in the form of four racemic pairs, i.e., two cis epimers and two trans epimers. A process for preparing such compounds is disclosed in, for example, Tetrahedron letters, 24, 5339-5342. In this process, cis 2-carboxyperhydroindole is synthesized from cyclohexanone and acrylonitrile as generally shown in Scheme I.

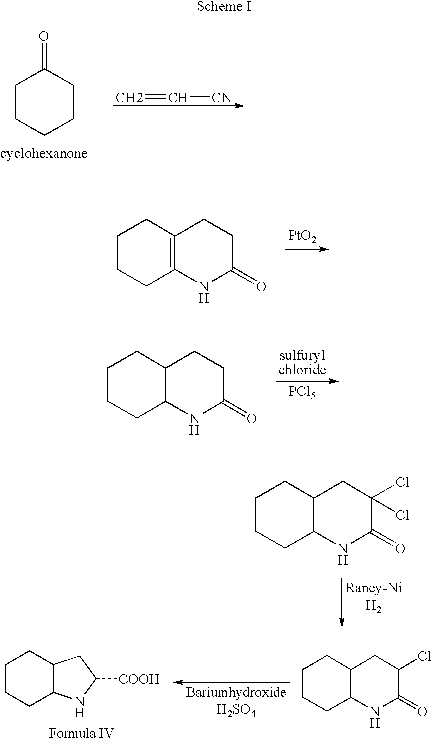

However, the isomer employed specifically in the synthesis of the compound of Formula I is (2S,3aS,7aS)-perhydroindole-2-carboxylic acid, as well as its esters, of Formula III.

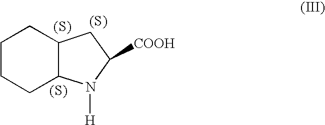

### SUMMARY OF THE INVENTION

In accordance with one embodiment of the present invention, a process for the preparation of (2S,3aS,7aS)perhydroindole-2-carboxylic acid of Formula III is provided: 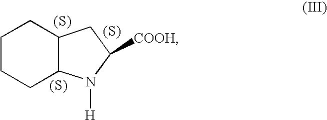
the process comprising:

(a) esterifying cis-perhydroindole-2-carboxylic acid of Formula IV: 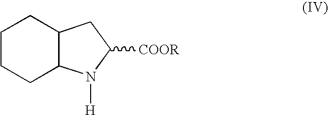
with a first alcohol of the formula ROH and a suitable free acid to provide the acid salt (AS) of formula V: 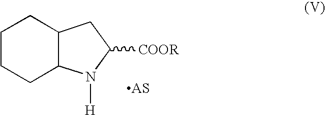
wherein R is a benzyl, alkyl, aryl or aralkyl group;

(b) reacting the acid salt of Formula V with a first base to provide a compound of Formula VI:

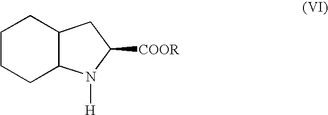

(c) treating the product of step (b) with an L-tartaric containing acid in a second alcohol of the formula ROH to precipitate a compound of Formula VII: 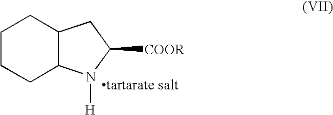
wherein R is a benzyl, alkyl, aryl or aralkyl group;

(d) reacting the compound of Formula VII with a second base to provide a compound of Formula II

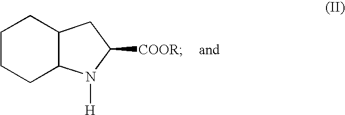

(e) hydrolyzing the compound of Formula II to provide the compound of Formula III.

The advantages of the present invention include at least:

1. The use of cis-perhydroindole-2-carboxylic acid as a starting material, which is readily available, cost effective and does not require arduous separation of carboxyperhydroindole isomers.

2. The process does not require the use of expensive catalyst for debenzylation.

3. The process provides relatively high yields with a relatively short process.

### DETAILED DESCRIPTION OF THE PREFERRED EMBODIMENTS

The present invention is directed to a process for the preparation of an intermediate of perindopril, (2S,3aS,7aS)perhydroindole-2-carboxylic acid of Formula III.

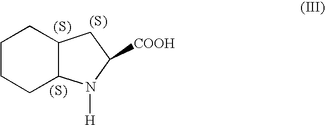

In step (a) of the process of the present invention, cis-perhydroindole-2-carboxylic acid of Formula IV: 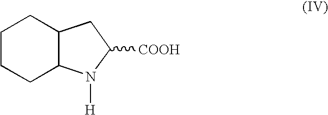
is esterified with a first alcohol of the formula ROH and a suitable free acid to provide the acid salt (AS) of formula V: 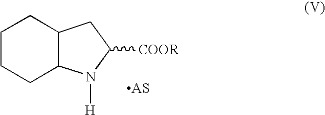
wherein R is a benzyl, alkyl, aryl or aralkyl group. The term alkyl as used herein refers to a saturated straight, branched, or cyclic, primary, secondary, or tertiary hydrocarbon of C₁-C₁₀. Representative examples of such alkyl groups includes, but are not limited to, methyl, ethyl, propyl, isopropyl, butyl, isobutyl, t-butyl, pentyl, cyclopentyl, isopenty), neopentyl, hexyl, isohexyl, cyclohexyl, cyclohexylmethyl, 3-methylpentyl, 2,2-dimethylbutyl, and 2,3-dimethylbutyl. The term “lower alkyl”, as used herein and unless otherwise specified, refers to a C₁-C₄ saturated straight or branched alkyl group. The term “aryl” as used herein refers to phenyl, biphenyl, or naphthyl, and preferably phenyl. The aryl group can be optionally substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art. The term “aralkyl” or “arylalkyl” refers to an aryl group with an alkyl substituent such as, for example, benzyl alcohol. Suitable free acids include, but are not limited to, p-toluenesulfonic acid, hydrochloric acid and the like and mixtures thereof.

In one embodiment, the cis-perhydroindole-2-carboxylic acid of Formula IV is esterified with benzyl alcohol in p-toluenesulfonic acid to provide the p-toluenesulfonic acid salt (PTSA) of Formula V: 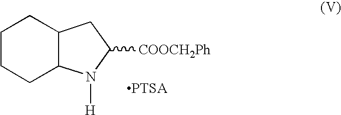
wherein Ph is phenyl.

In step (b) of the process of the present invention, the acid salt of Formula V is reacted with a base to provide a compound of Formula VI.

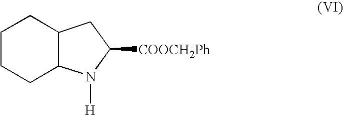

In one embodiment, the base of step (b) is an organic amine base. Suitable amine bases for use in the first step of the present invention include, but are not limited to, primary amines, secondary amines, tertiary amines, aliphatic amines, aromatic amines, ammonia and the like and mixtures thereof. In one embodiment, the amine base is a tertiary amine, heterocyclic amine and mixtures thereof. In another embodiment, the amine base is a trialkylamine, heterocyclic amine and mixtures thereof. Useful tertiary amine base groups include, but are not limited to, tri(lower alkyl)amines containing from about 3 to about 20 carbon atoms such as trimethylamine, triethylamine, tripropylamine, tributylamine and the like and mixtures thereof. Useful heterocyclic amine base groups include, but are not limited to, substituted and unsubstituted pyridines, substituted and unsubstituted morpholines, substituted and unsubstituted piperazines, substituted and unsubstituted piperidines, substituted and unsubstituted pyrrolidines, and the like and mixtures thereof. In another embodiment of the present invention, the amine base employed in the first step is selected from the group consisting of triethylamine and ammonia. In another embodiment, the base of step (b) includes, but is not limited to, alkali metal or alkali earth metal carbonates such as sodium carbonate and potassium carbonate and the like and mixtures thereof.

In step (c) of the process of the present invention, an L-tartaric containing acid such as dibenzoyl L-tartaric acid, L-tartaric acid and the like in an alcohol of the formula ROH is added to the product of step (b) to form a solution and precipitate a compound of Formula VII: 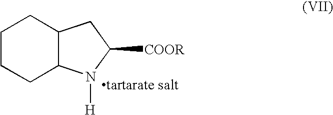
wherein R is a benzyl, alkyl, aryl or aralkyl group as described above. Step (c) involves the first resolution with the L-tartaric containing acid. Suitable alcohols include, but are not limited to, methanol, ethanol, butanol, propanol, isopropyl alcohol, benzyl alcohol and the like and mixtures thereof.

In step (d) of the process of the present invention, the compound of Formula VII is reacted with a base to provide a compound of Formula II.

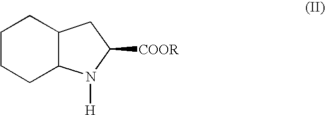

A useful base includes, but is not limited to, alkali metal hydroxides, alkali earth metal hydroxides and the like and mixtures thereof. Suitable alkali metal hydroxides include, but are not limited to, sodium hydroxide, potassium hydroxide and the like and mixtures thereof.

In step (e) of the process of the present invention, the compound of Formula II is hydrolyzed to provide a compound of Formula III. The compound of Formula II can be hydrolyzed with, for example, an alkali metal hydroxide.

In accordance with another aspect of the present invention, the compound of Formula III is thereafter converted to perindopril or a derivative thereof or a pharmaceutically acceptable salt thereof. In one embodiment, the compound of Formula III is thereafter converted to perindopril erbumine

The following examples are provided to enable one skilled in the art to practice the invention and are merely illustrative of the invention. The examples should not be read as limiting the scope of the invention.

### EXAMPLE 1

Preparation of (2S,3aS,7aS)-perhydroindole-2-carboxylic acid.

A mixture of cis-perhydroindole-2-carboxylic acid (150 g), toluene [1.5 lit], p-toluenesulfonic acid (202.6 g) and benzyl alcohol (162.95 g) were mixed in a vessel. The mixture was heated to reflux and the water was removed by azeotropic distillation. After removal of the water, the reaction mixture was refluxed for an additional 3 hours. Following completion of the reaction as determined by TLC, the reaction mixture was slowly cooled to a temperature of about 25° C. The product was filtered off and washed with toluene (300 ml) to yield benzyl-perhydroindole-2-carboxylate PTSA salt (300 g). The benzyl-perhydroindole-2-carboxylate PTSA salt (300 g) was charged with methylene dichloride (3 L). Triethylamine (193.9 ml) was then added and the mixture was stirred for about 30 minutes. The organic phase was separated and washed with water twice (1.5 L×2). The organic layer was then concentrated to yield benzyl-perhydroindole-2-carboxylate (180 g) as an oil.

The above obtained oil was taken in methanol (0.9 L) and a solution of dibenzoyl-L-tartaric acid (249 g) in methanol (0.9 L) was added at a temperature of about 25° C. The reaction mixture was stirred for about 30 minutes and then heated to a temperature of about 60° C for about 1 hour. The reaction mixture was cooled to a temperature of about 15° C. The solid was filtered off and washed with methanol (180 ml). The solid was dried at a temperature of about 60° C. to yield the tartarate salt of benzyl-perhydroindole-2-carboxylate (180 g).

The benzyl-perhydroindole-2-carboxylate tartarate salt (180 g) was added to methylene chloride (1.8 L) and charged with a solution of sodium hydroxide (23.4 g) in water (0.9 L). The reaction mixture was stirred for about 1 hour. The organic layer was separated and washed with water (900 ml) and then with a brine water solution (900 ml). The organic phase was separated and the methylene chloride was removed to yield (2S,3aS,7aS)-benzyl-perhydroindole-2-carboxylate (72 g).

The (2S,3aS,7aS)benzyl-perhydroindole-2-carboxylate (70 g) was added to methanol (350 ml) and charged with a solution of sodium hydroxide (13.4 g) in water (50 ml). The reaction mixture was refluxed for about 2 hours. After completion of reaction as determined by TLC, the pH of the reaction mixture was adjusted to a range of from about 6 to about 7 with dilute hydrochloric acid. The methanol was concentrated and charged with ethanol (700 ml). The reaction mixture was heated to a temperature of about 70° C. and the inorganics were filtered off. The ethanol was concentrated and the product was isolated with acetone (700 ml). The product was dried at a temperature of about 60° C. to yield (2S,3aS,7aS)-perhydroindole-2-carboxylic acid (42 g).

### EXAMPLE 2

Preparation of N-[1-(S)-ehoxycarbonyl-1-butyl]-(S)-alanyl chloride hydrochloride.

Into a 4 necked round bottom (RB) flask, methylene chloride (700 ml) and phosphorous pentachloride (148 g) were added and stirred for 90 minutes at 20 to 25° C. The mixture was cooled to −10° C. and N-i(S)-carboxyethylbutyl-(S)-alanine (100 g) was added and maintained for 5 hours at −5 to 0° C. Diisopropyl ether (2 1) was slowly added while maintaining the temperature below −5° C. and then at −5 to 0° C. for 1 hour. The solid was filtered under nitrogen atmosphere and dried at 40-45° C. under vacuum. Dry wt. 122 grams.

### EXAMPLE 3

Preparation of Perindopril tert-butyl amine salt.

Into a 4 necked RB flask, methylene chloride (700 ml) and N-1(S)-carboxyethylbutyl-(S)-alanyl chloride hydrochloride (62g) from Example 2 were added and cooled to about −5° C. Imidazole (56.4 g) was added to the mixture and maintained for 1 hour at below 0° C. Next, (2S,3aS,7aS)-2-carboxyperhydroindole (35 g) from Example 1 was added slowly in about 45 minutes. The reaction mass was stirred for about 2 hours at a temperature about −5 to 0° C. and then raised to 20 to 25° C. for 2 hours. The mixture of acetic acid (37 g) in water (350 ml) was added by maintaining the temperature below 5° C. and stirred for 30 minutes. The methylene chloride layer was separated, washed with saturated brine solution (70 ml) and dried over sodium sulfate (3.5 g).

The methylene chloride layer was charged in a RB flask and cooled to 10° C. Next, tert-butyl amine (23 ml) was charged in the flask for 30 minutes by maintaining the temperature below 10° C. and stirred further for 30 minutes at 35-40° C. Methylene chloride was distilled off completely and a mixture of isopropyl alcohol(100 ml), acetone (200 ml) and acetonitrile (200 ml) was charged in the flask and heated to about 65-70° C. to provide a clear solution. The reaction mass was then cooled very slowly to about 25° C. in 2 hours and then further cooled to about 5-10° C. and filtered. The material was then dried under vacuum at about 40° C. (Weight: 43 g, purity: >99.5% by HPLC). Specific optical rotation [α]n =-66 (C=1%, Methanol), IR (KBr) spectrum shows the following absorptions cm-1 3300, 2930, 1744, 1732m 1644m 1568. The 1H-NMR (CDC13) shows the following signals at δ4.28-4.12 (m, 1H), 4.18-4.09 (q,2H), 3.76 (m,2H) 3.53 (q, 1H), 3.1 (t, 1H), 2.32-2.14 (m, 2H), 2.01 (m, 1H), 1.75-1.62 (m, 4H), 1.32 (m,2H) 3.53 (S, 9H), 1.28 (t, 3H), 0.88 (t, 3H). C.I. Mass shows m/z at 368 (base peak.)

### EXAMPLE 4

Preparation of Perindopril tert-butyl amine salt

Into a 4 necked RB flask, methylene chloride (50 ml) and (2S,3aS,7aS)-2-carboxyperhydroindole (5 gm) from Example 1 were added and cooled to about 10° C. Next, trimethyl silyl chloride (3.21 gm) was added dropwise while maintaining the temperature below 10° C. and stirred for 10 minutes. Imidazole (2 gm) was added maintaining the temperature below 10° C. The reaction mass was then stirred for about 2 hours at a temperature of about 10-15 ° C. to provide the trimethyl silyl ester of (2S,3aS,7aS)-2-carboxyperhyd Into another 4 necked RB flask, methylene chloride (100 ml) and N-1(S)-carboxyethylbutyl-(S)-alanyl chloride (8.85 gm) were added and cooled to about 10° C. Imidazole (8.04 gm) was added at below 10° C. and maintained for 1 hour at 10-15° C. The trimethyl silyl ester of (2S,3aS,7aS)-2-carboxyperhydroindole was then added dropwise slowly in 1 hour at 10° C. The reaction mixture was stirred for 2 hours at 10° C.

A mixture of acetic acid (5.32 gm) in methylene chloride (20 ml) was added to the reaction mixture maintaining the temperature below 5° C. and stirred for 30 minutes. Water (30 ml) was added and stirred for 30 minutes. The methylene chloride layer was separated, washed with saturated brine solution (15 ml) and dried over sodium sulfate. The methylene chloride layer was charged in a RB flask and cooled to 5° C. Tert-butyl amine (3.25 ml) was charged in 30 minutes by maintaining the temperature below 10° C. and stirred further for 30 minutes at 35-40° C. Methylene chloride was then distilled off completely and a mixture of isopropyl alcohol (15 ml), acetone (30 ml) and acetonitirle (30 ml) was charged and heated to about 65-70° C. to provide a clear solution. The reaction mass was cooled very slowly to about 25° C. in 2 hours and then further cooled to about 5-10° C. and filtered. The material was then dried under vacuum at about 40° C. (Weight: 7.5 g, purity: >99.5% by HPLC). Specific optical rotation [α]n=-66 (C=1%, MeOH), IR (KBr) spectrum shows the following absorptions cm-1 3300, 2930, 1744, 1732m 1644m 1568. The 1H-NMR (CDC13) shows the following signals at δ4.28-4.12 (m, 1H), 4.18-4.09 (q,2H), 3.76 (m,2H) 3.53 (q,1H), 3.1 (t,1H), 2.32-2.14 (m,2H), 2.01 (m,1H), 1.75-1.62 (m,4H), 1.32 (m,2H), 1.30 (S,9H), 1.28 (t, 3H), 0.88 (t, 3H). CI Mass shows m/z at 368 (base peak.)

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention.

What is claimed is:

1. A process for the preparation of (2S,3aS,7aS)perhydroindole-2-carboxylic acid of Formula III 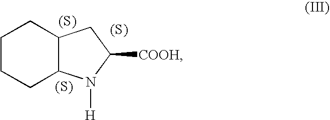
the process comprising:
(a) esterifying cis-perhydroindole-2-carboxylic acid of Formula IV: 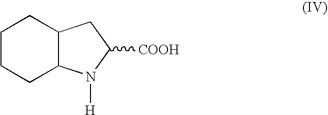
with a first alcohol of the formula ROH and a suitable free acid to provide the acid salt (AS) of formula V: 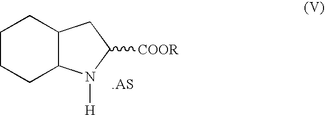
wherein R is an alkyl, aryl or aralkyl group;
(b) reacting the acid salt of Formula V with a first base to provide a compound of Formula VI: 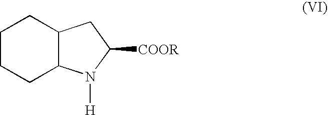
(c) treating the product of step (b) with an L-tartaric containing acid in a second alcohol of the formula ROH to precipitate a compound of Formula VII: 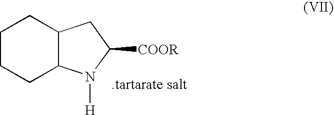
wherein R has the aforestated meaning;
(d) reacting the compound of Formula VII with a second base to provide a compound of Formula II 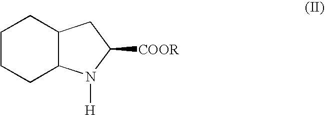
wherein R has the aforestated meaning; and
(e) hydrolyzing the compound of Formula II to provide the compound of Formula III.

2. The process of claim 1, wherein the first alcohol is benzyl alcohol.

3. The process of claim 1, wherein the first alcohol is selected from the group consisting of methanol, ethanol, isopropanol, propanol, butanol and mixtures thereof.

4. The process of claim 1, wherein the free acid is selected from the group consisting of p-toluenesulfonic acid, hydrochloric acid and mixtures thereof.

5. The process of claim 1, wherein the first base is organic amine base.

6. The process of claim 1, wherein the first base is selected from the group consisting of a primary amine, secondary amine, tertiary amine, aliphatic amine, aromatic amine, ammonia and mixtures thereof.

7. The process of claim 1, wherein the first base is selected from the group consisting of a tertiary amine, heterocyclic amine and mixtures thereof.

8. The process of claim 1, wherein the first base is selected from the group consisting of a trialkylamine, heterocyclic amine and mixtures thereof.

9. The process of claim 8, wherein the trialkylamine contains from about 3 to about 20 carbon atoms.

10. The process of claim 1, wherein the first base is selected from the group consisting of trimethylamine, triethylamine, tripropylamine, tributylamine and mixtures thereof.

11. The process of claim 1, wherein the first base is selected from the group consisting of an alkali metal carbonate, alkali earth metal carbonate and mixtures thereof.

12. The process of claim 1, wherein the first base is selected from the group consisting of sodium carbonate, potassium carbonate and mixtures thereof.

13. The process of claim 1, wherein the L-tartaric containing acid is dibenzoyl L-tartaric acid or L-tartaric acid.

14. The process of claim 1, wherein the second alcohol is benzyl alcohol.

15. The process of claim 1, wherein the second alcohol is selected from the group consisting of methanol, ethanol, isopropanol, propanol, butanol and mixtures thereof.

16. The process of claim 1, wherein the second base is selected from the group consisting of an alkali metal hydroxide, alkali earth metal hydroxide and mixtures thereof.

17. The process of claim 1, wherein the second base is selected from the group consisting of sodium hydroxide, potassium hydroxide and mixtures thereof.

18. The process of claim 1, wherein in step (e) the compound of Formula II is hydrolyzed with an alkali metal hydroxide.

19. The process of claim 1, wherein the compound of Formula III is thereafter converted to perindopril or a pharmaceutically acceptable salt thereof.

20. Perindopril erbumine obtained from the process of claim 19.
